(19) United States
(12) Patent Application Publication
(10) Pub. No.: US 2004/0203012 A1
(43) Pub. Date: Oct. 14, 2004

(54) Method of detecting and monitoring prostate and ovarian cancers

(76) Inventor: Eleftherios Diamandis, Toronto (CA)

Correspondence Address: Merchant & Gould, PO Box 2903, Minneapolis, MN, 55402-0903, US

(21) Appl. No.: 10/478,333

(22) PCT Filed: May 24, 2002
(86) PCT No.: PCT/CA02/00770

(30) Foreign Application Priority Data
May 25, 2001 (US) 60293482
Sep. 24, 2001 (US) 60324350

(51) Int. Cl. C12Q 1/68; G01N 33/574

(52) U.S. Cl. 435/6; 435/7.23

(57) ABSTRACT

A method for the diagnosis, prognosis, and monitoring of ovarian or prostate cancer in a subject by detecting hK11 in a sample from the subject, preferably a serum sample. hK11 may be measured using a reagent that detects or binds to hK11 preferably antibodies specifically reactive with hK11 or a part thereof. Imaging methods for tumors associated with hK11 are also described using an agent that binds to hK11 which has a label for imaging the tumor.

### FIELD OF THE INVENTION

The invention relates to diagnostic methods for cancer.

### BACKGROUND OF THE INVENTION

Prostate-specific antigen (PSA) is the best described cancer marker and it is currently used widely for diagnosis and monitoring of prostatic carcinoma (12). PSA is a member of the human kallikrein family of serine proteases and has chymotrypsin-like enzymatic activity. Another member of the human kallikrein family, human glandular kallikrein 2 (hK2) is a new potential prostatic biomarker (5). Recently, the human kallikrein gene family has been expanded to include 15 members which share significant similarities at both the DNA and amino acid level (6, 25). All members of the human kallikrein gene family localize on chromosome 19q13.4 and encode for secreted serine proteases. Recently, it has been reported that two members of this family, hK6 and hK10, are potential biomarkers for diagnosis and monitoring of prostate or ovarian cancer (26,27). In addition, many other members of the same family have been found to be overexpressed or underexpressed in ovarian and other cancers (28-37).

The gene encoding hK11 was first cloned by Yoshida et al. and was named trypsin-like serine protease (8). With the newly established kallikrein gene nomenclature, this gene is now known as human kallikrein 11 (KLK11; the protein is designated as hK11)(7). No methods currently exist for measuring hK11 protein in tissue extracts or biological fluids. By reverse transcription-polymerase chain reaction (RT-PCR), it was demonstrated that the KLK11 gene is expressed in many tissues, including brain, skin, salivary gland, stomach, prostate and intestine (9).

### SUMMARY OF THE INVENTION

Applicant found that the hK11 concentration in serum is significantly elevated in patients with cancer, particularly prostate cancer and ovarian cancer, in comparison with normal subjects. Thus, hK11 constitutes a new biomarker for diagnosis, monitoring (i.e. monitoring progression or therapeutic treatment), and prognosis of cancer. In particular, hK11 may be used for the diagnosis, monitoring, and prognosis of prostate or ovarian cancer, and it may be used as a biomarker before surgery or after relapse.

hK11, and agents that bind to hK11 may be used to detect ovarian or prostate cancer and they can be used in the diagnostic evaluation of ovarian or prostate cancer, and the identification of subjects with a predisposition to such disorders.

The term “detect” or “detecting” includes assaying, imaging or otherwise establishing the presence or absence of the target hK11, subunits thereof, or combinations of reagent bound targets, and the like, or assaying for, imaging, ascertaining, establishing, or otherwise determining one or more factual characteristics of ovarian or prostate cancer, metastasis, stage, or similar conditions. The term encompasses diagnostic, prognostic, and monitoring applications for hK11.

Methods for determining the presence of hK11 can be used to monitor ovarian or prostate cancer by detecting or using hK11.

The present invention relates to a method for diagnosing and monitoring of prostate or ovarian carcinoma in a subject comprising measuring hK11 in a sample from the subject. hK11 may be measured using a reagent that detects hK11 or a nucleic acid sequence encoding hK11.

In an aspect of the invention a method is provided for detecting the expression of the cancer marker hK11 in a subject which comprises taking a sample derived from a subject, and detecting in the sample a nucleic acid sequence encoding hK11 or a protein product encoded by a hK11 nucleic acid sequence.

In an aspect of the invention, a method for screening a subject for ovarian or prostate cancer is provided comprising (a) obtaining a biological sample from a subject; (b) detecting the amount of hK11 in said sample; and (c) comparing said amount of hK11 detected to a predetermined standard, where detection of a level of hK11 that is different (e.g. greater) than that of a predetermined standard indicates disease.

In another aspect the present invention provides a diagnostic method for detecting the presence of prostate or ovarian cancer in a subject suspected of suffering from prostate or ovarian cancer which comprises: (a) measuring levels of hK11 in a biological sample (e.g. cells, tissues or bodily fluids) obtained from a subject suspected of suffering from prostate or ovarian cancer; and (b) comparing the measured levels of hK11 polypeptide with levels of hK11 in a predetermined standard (e.g. normal control) where detection of a level of hK11 different (e.g. greater) than that of a standard indicates disease.

A predetermined standard may correspond to levels determined for samples from normal control subjects without cancer (e.g. normal cells, tissues or bodily fluids), from subjects with a different disease stage, or from other samples of the subject. Increased hK11 polypeptide levels in the subject compared to a standard that is a normal control is typically indicative of prostate or ovarian cancer.

The present invention also provides a diagnostic method for detecting the presence of prostate or ovarian cancer in a subject suspected of suffering from prostate or ovarian cancer which comprises: (a) measuring transcription levels of hK11 in a biological sample (e.g. cells, tissues or bodily fluids) from a subject suspected of suffering from prostate or ovarian cancer; and (b) comparing the measured transcription levels of hK11 with hK11 transcription levels in a biological sample (e.g. normal cells, tissues or bodily fluids) from a predetermined standard (e.g. normal control), wherein a change (e.g. an increase) in hK11 transcription levels in the subject versus hK11 transcription levels in the predetermined standard (e.g. normal control) is associated with prostate or ovarian cancer.

Still further the present invention provides a method of monitoring in a subject prostate or ovarian cancer for the onset of metastasis which comprises: (a) identifying a subject suffering from prostate or ovarian cancer that is not known to have metastasized; (b) measuring hK11 levels in a biological sample from the subject; and (c) comparing the measured hK11 levels in the subject with levels of hK11 in the same type of biological sample from a predetermined standard (e.g. normal control), wherein a change (e.g. an increase)in measured hK11 levels in the subject versus hK11 levels in the predetermined standard (e.g. normal control) is associated with a cancer which has metastasized.

Another aspect of the present invention provides a method of monitoring the stage of prostate or ovarian cancer in a subject suffering from prostate or ovarian cancer which comprises: (a) identifying a subject suffering from prostate or ovarian cancer; (b) determining hK11 levels in a biological sample from the subject to establish a baseline hK11 level for the subject; (c) measuring hK11 levels in biological samples of the same type from the subject at subsequent time periods; and (d) comparing the measured hK11 levels with the baseline hK11 levels, wherein an increase in measured hK11 levels in the subject versus baseline hK11 levels in the subject is associated with a cancer which is progressing, and a decrease in measured hK11 levels versus baseline hK11 levels is associated with a cancer which is regressing or in remission.

In an embodiment of the invention hK11 is measured using a substance that binds with the hK11, preferably an antibody specific for hK11.

Therefore, the invention relates to a method for detecting prostate or ovarian carcinoma in a subject by quantitating hK11 in a biological sample from the subject comprising: (a) reacting the biological sample with an antibody specific for hK11 which is directly or indirectly labelled with a detectable substance; (b) detecting the detectable substance to determine the levels of hK11 in the biological sample; and (c) comparing the levels of hK11 with levels of hK11 in biological samples from a predetermined standard (e.g. normal control) wherein a change (e.g. an increase) in hK11 levels is indicative of prostate or ovarian cancer.

Embodiments of the methods of the invention involve (a) reacting a biological sample from a subject with an antibody specific for hK11 which is directly or indirectly labelled with an enzyme; (b) adding a substrate for the enzyme wherein the substrate is selected so that the substrate, or a reaction product of the enzyme and substrate forms fluorescent complexes;(c) quantitating hK11 in the biological sample by measuring fluorescence of the fluorescent complexes; and (d) comparing the quantitated levels to levels obtained for a predetermined standard [e.g. other biological samples from the subject patient (such as biological samples taken previously or subsequently), or from a control subject]. In an embodiment, the quantitated levels are compared to levels quantitated for subjects without prostate or ovarian carcinoma (i.e. normal controls) wherein an increase in hK11 levels compared with the control subjects is indicative of prostate or ovarian carcinoma.

A preferred embodiment of the invention comprises the following steps

(a) incubating a biological sample with a first antibody specific for hK11 which is directly or indirectly labeled with a detectable substance, and a second antibody specific for hK11 which is preferably immobilized;

(b) separating the first antibody from the second antibody to provide a first antibody phase and a second antibody phase;

(c) detecting the detectable substance in the first or second antibody phase thereby measuring hK11 levels in the biological sample; and

(d) comparing the measured hK11 levels with levels measured for biological samples from a predetermined standard [e.g. normal controls or from other biological samples of the subject (e.g. prior or subsequent biological samples)].

The invention also contemplates the methods described herein using multiple markers for ovarian or prostate cancer. Therefore, the invention contemplates a method for anaylzing a biological sample for the presence of hK11 and other markers that arc specific indicators of ovarian or prostate cancer. Other ovarian markers include markers such as human stratum corneum chymotryptic enzyme (HSCCE), kallikrein 2, kallikrein 3, kallikrein 4, kallikrein 5, KLK5 gene, kallikrein 6, kallikrein 8, kallikrein 9, KLK9 gene, kallikrein 10, KLK10 gene, kallikrein 15, and KLK15 gene; CA125, CA15-3, CA19-9, OVX1, lysophosphatidic acid (LPA) and carcinoembryonic antigen (CEA). Preferably the other markers are markers to kallikreins. In an aspect of the invention, the other ovarian cancer markers are one or more of KLK4, hK6, hK8, KLK8, hK9, KLK9, hK10, KLK10, and CA125. Other prostate cancer markers include hK2; hK3, hK4, hK6, hK10, KLK5, KLK10,HER-2, KLK15 and prostate-specific antigen. The methods described herein may be modified by including reagents to detect the markers, or nucleic acids for the markers.

In accordance with an aspect of the invention an in vivo method is provided comprising administering to a subject an agent that has been constructed to target hK11 and one or more other kallikreins.

The invention contemplates an in vivo method comprising administering to a mammal one or more agent that carries a label for imaging and binds to a hK11, and then imaging the mammal.

According to a preferred aspect of the invention, an in vivo method for imaging ovarian or prostate cancer is provided comprising:

(a) injecting a patient with an agent that binds to kallikrein 11, the agent carrying a label for imaging the ovarian or prostate cancer;

(b) allowing the agent to incubate in vivo and bind to kallikrein 11 associated with the ovarian or prostate cancer; and

(c) detecting the presence of the label localized to the ovarian or prostate cancer.

In an embodiment of the invention the agent is an antibody which recognizes the kallikrein 11. In another embodiment of the invention the agent is a chemical entity which recognizes the kallikrein 11.

In an aspect of the invention, a method is provided for localizing ovarian or prostate cancer cells or tumors in a subject by administering an antibody specific for hK11, allowing the antibody to bind to hK11 in the cancer cells within the subject, and determining the location of the antibody within the patient. In another related aspect, the antibody is detectably labeled, for example with a radiolabel.

The agent carries a label to image the kallikreins. Examples of labels useful for imaging are radiolabels, fluorescent labels (e.g. fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for ovarian cancer. For example, a method for imaging ovarian cancer may further comprise injecting the patient with one or more of an agent that binds to human stratum corneum chymotryptic enzyme (HSCCE), kallikrein 4, kallikrein 5, kallikrein 6, kallikrein 8, kallikrein 9, kallikrein 10, kallikrein 15, CA125, CA15-3, CA19-9, OVX1, lysophosphatidic acid (LPA) or carcinoembryonic antigen (CEA), preferably CA125. A method for imaging prostate cancer may further comprise injecting the patient with one or more of an agent that binds to kallikrein 2, kallikrein 5, kallikrein 10, kallikrein 15, HER-2, or prostate-specific antigen. Nucleic acid molecules may also be detected for these other markers.

The invention also relates to kits for carrying out the methods of the invention.

The invention is also directed to methods for inhibiting or killing ovarian or prostate cancer cells in a subject by administering an antibody specific for hK11 under conditions sufficient for the antibody to inhibit or kill the cells. In another aspect, a method for inhibiting or killing ovarian or prostate cancer cells is provided comprising administering an antibody specific for hK11 that is conjugated with a cytotoxic moiety, under conditions sufficient for the antibody to inhibit or kill the cells. The cytotoxic moiety may be, by way of non-limiting example, a chemotherapeutic agent, a photo-activated toxin, or radioactive agent.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1: Calibration curve of an hK11 immunofluorometric assay. The assay has a dynamic range of 0.1 μg/L (lowest detection limit) to 50 μg/L. The fluorescence of the zero standard was subtracted from all other measurements.

FIG. 2: Human kallikrein 11 (hK11) content of cytosolic extracts of various human tissues. All hK11 concentrations were corrected for the amount of total protein.

FIG. 3: Immunohistochemical localization of hK11 protein in tissues. Staining was performed with a monoclonal anti-hK11 antibody, as described in Example 1. (A) Intense staining in the supranuclear cytoplasm of small intestinal epithelial cells (original magnification ×400). (B) Another section of the same tissue as in A. (C) Immunohistochemical staining of hK11 in epithelial cells of invasive papillary serous carcinoma of ovary (original magnification ×400). (D) Another section of the same tissue as in (C).

FIG. 4: Hormonal regulation of hK11 production in the breast carcinoma cell lines BT-474 and MCF-7. Highest hK11 protein concentration in tissue culture supernatants was seen in cells treated with estradiol. In BT474 cells, highest levels of PSA in tissue culture supernatants were seen after induction with dihydrotestosterone and norgestrel, as previously described (23). Abbreviations: AL, alcohol (negative control); aldo, aldosterone; estra, estradiol; dexa, dexamethasone; DHT, dihydrotestosterone; norg, norgestrel.

FIG. 5: Distribution of hK11 concentration in serum of 40 females (A) and 32 males (B). For medians see Example 1.

FIG. 6: High performance liquid chromatographic separation on a gel filtration column of a serum sample (A) and a seminal plasma sample (B). In serum, a major peak around 33 kDa is shown along with a small peak around 100 kDa. The major peak represents the free form of hK11. In seminal plasma, only the free form of this enzyme is detectable.

### DETAILED DESCRIPTION OF THE INVENTION

The invention relates to methods, both quantitative and qualitative for detecting levels of hK11 polypeptide or nucleic acids in biological samples, including determination of normal and abnormal levels. Diagnostic methods in accordance with the invention for detecting over-expression of an hK11 polypeptide compared to normal controls via detection of polypeptide or transcription levels may be used to detect the presence of cancers, including prostate and ovarian cancer.

By “hK11”, “hK11 protein”, or “hK11 polypeptide” is meant a protein or fragment thereof having an amino acid sequence identical to or substantially similar to that disclosed for hK11 in Genbank Accession Nos. XM009005 and AB012917, and Yoshida et al (8). Polypeptides which are “substantially similar” to the hK11 protein disclosed in Genbank Accession Nos. XM009005 and AB012917, and Yoshida et al (8) may contain conservative amino acid substitutions which do not alter the structure or activity of the hK11 protein. When the diagnostic method of the invention is used to diagnosis or monitor cancer in a species other than a human, the term “hK11” used herein includes kallikrein 11 from that species. The term also includes all homologs, naturally occurring allelic variants, isoforms and precursors of human kallikrein 11. In general for example, naturally occurring allelic variants of human kallikrein 11 will share significant homology (70-90%) to the sequences shown in GenBank Accession Nos. XM009005 and AB012917. hK11 fragments are preferably biologically active i.e. exert the biological or physical effects of the full-length hK11 protein.

“hK11 nucleic acids” or “KLK11” is meant to include both RNA and DNA encoding the hK11 protein as disclosed in Genbank Accession Nos. XM009005 and AB012917, and Yoshida et al (8)or a polypeptide with the same structure and activity.

The term “subject” refers to a warm-blooded animal such as a mammal that is afflicted with prostate or ovarian cancer or condition as described herein. Preferably, “subject” refers to a mammal, most preferably a human.

The terms “sample”, “biological sample”, and the like mean a material known or suspected of expressing or containing hK11. The test sample can be used directly as obtained from the source or following a pretreatment to modify the character of the sample. The sample can be derived from any biological source, such as tissues, bodily fluids, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. Therefore, a biological sample may be blood, urine, saliva, a tissue biopsy, or autopsy material. Preferably, the sample is serum. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

The methods described herein can be adapted for diagnosing and monitoring prostate or ovarian carcinoma by quantitating hK11 in biological samples from a subject. These applications require that the amount of hK11 quantitated in a sample from a subject being tested be compared to predetermined standards, for example, levels quantitated for another sample or an earlier sample (e.g. baseline hK11 levels) from the subject, or levels quantitated for a control sample. Levels for control samples from normal or healthy subjects may be established by prospective and/or retrospective statistical studies. Healthy subjects who have no clinically evident disease or abnormalities may be selected for statistical studies. Diagnosis may be made by a finding of statistically different levels of hK11 compared to a normal control sample or previous levels quantitated for the same subject. Generally, elevated levels of hK11 measured in a subject compared with levels in a normal control are indicative of prostate or ovarian cancer. “Elevated levels” generally refers to greater than the 90^{th} to 95^{th} percentile, preferably 95^{th} percentile, of normal controls.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The monitoring of hK11 levels in a subject diagnosed with prostate or ovarian cancer is useful in determining the onset of metastases in cancers that have not yet metastasized. In this method a subject suffering from prostate or ovarian cancer that is not known to have metastasized is identified. hK11 levels in a biological sample from the subject are measured and compared with levels of hK11 in the same type of biological sample from a normal control or subject with metastases. A change in measured hK11 levels in the patient versus the standard is associated with a cancer that has metastasized.

The stage of prostate or ovarian cancer in a subject suffering from prostate or ovarian cancer cart also be determined. hK11 levels in a biological sample from the subject are measured to establish a baseline hK11 level for the subject. hK11 levels in the same type of biological sample are then determined at subsequent periods such as scheduled check-ups with a physician. Measured hK11 levels are compared with the baseline hK11 levels for the subject. In this method a decrease in measured hK11 levels in the subject versus baseline hK11 levels in the subject is associated with a cancer which is regressing or in remission. Increases in measured hK11 levels as compared to baseline hK11 levels established for the subject may be indicative of disease progression or metastases.

Methods that can be used to determine levels of a polypeptide or transcription levels of a gene such as hK11, in a biological sample derived from a subject are well-known to those skilled in the art. A variety of methods can be employed for the diagnostic and prognostic evaluation of ovarian or prostate cancer involving hK11, and the identification of subjects with a predisposition to such disorders. Examples of these methods include but are not limited to radioimmunoassays, reverse transcriptase PCR (RT-PCR) assays, gridding, immunohistochemistry assays, in situ hybridization assays, competitive-binding assays, Western Blot analyses, and ELISA assays. Assays using antibodies (or derivatives thereof) specific for a protein are frequently preferred to detect the protein in biological fluids. Methods may, for example, utilize antibodies directed against hK11 including peptide fragments. In particular, the antibodies may be used, for example, for the detection of either an over- or an under-abundance of hK11 relative to a non-disorder state or the presence of a modified (e.g., less than full length) hK11 which correlates with a disorder state, or a progression toward a disorder state.

Antibodies specifically reactive with a hK11 protein, or derivatives, such as enzyme conjugates or labeled derivatives, may be used to detect hK11 protein in various samples. They may be used as diagnostic or prognostic reagents and they may be used to detect abnormalities in the level of hK11 expression, or abnormalities in the structure, and/or temporal, tissue, cellular, or subcellular location of hK11. In particular, they can be used to evaluate the probability of the presence of malignant or pre-malignant disease, and to determine the onset of metastases and disease stage. Antibodies may also be used to screen potentially therapeutic compounds in vitro to determine their effects on disorders (e.g. ovarian or prostate cancer) involving a hK11 protein, and other conditions. In vitro immunoassays may also be used to assess or monitor the efficacy of particular therapies. The invention also contemplates pharmaceutical compositions comprising an antibody of the invention, and a pharmaceutically acceptable carrier or diluent.

In an embodiment, the present invention provides a diagnostic method for monitoring or diagnosing prostate or ovarian carcinoma in a subject by quantitating hK11 in a biological sample from the subject comprising reacting the sample with an antibody specific for hK11 which is directly or indirectly labelled with a detectable substance, and detecting the detectable substance.

In another embodiment, the invention contemplates a method for monitoring the progression of ovarian or prostate cancer in an individual, comprising:

(a) contacting an antibody which binds to a hK11 protein, with a sample from the individual so as to form a complex comprising the antibody and hK11 protein in the sample;

(b) determining or detecting the presence or amount of complex formation in the sample;

(c) repeating steps (a) and (b) at a point later in time; and

(d) comparing the result of step (b) with the result of step (c), wherein a difference in the amount of complex formation is indicative of disease, disease stage, and/or progression of the ovarian or prostate cancer in said individual.

The amount of complexes may also be compared to a value representative of the amount of the complexes from an individual not at risk of, or afflicted with, ovarian cancer at different stages.

Antibodies specific for hK11 may be obtained from scientific or commercial sources. Alternatively, isolated native hK11 or recombinant hK11 may be utilized to prepare antibodies, monoclonal or polyclonal antibodies, and immunologically active fragments (e.g. a Fab or (Fab)₂ fragment), an antibody heavy chain, humanized antibody, an antibody light chain, a genetically engineered single chain F, molecule (Ladneret al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Preferably, antibodies used in the methods of the invention are reactive against hK11 if they bind with a Kₐ of greater than or equal to 10⁻⁷ M. In a preferred sandwich immunoassay of the invention mouse polyclonal antibodies and rabbit polyclonal antibodies are utilized.

Antibodies specifically reactive with hK11 may be used in any known immunoassays which rely on the binding interaction between an antigenic determinant of a protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify hK11 in a sample in order to diagnose and treat such pathological states.

An antibody specific for hK11 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against hK11. By way of example, if the antibody having specificity against hK11 is a rabbit IgG antibody, the second antibody may be goat anti-rabbit gamma-globulin labelled with a detectable substance as described herein.

An antibody specific for hK11 may be associated with a cytotoxic moiety. Suitable cytotoxic moieties include chemotherapeutic agents, photo-activated toxins, or radioactive agents. Examples of cytotoxic agents include but are not limited to ricin A chain, abrin A chain, modeccin A chain, gelonin, melphalan, bleomycin, adriamycin, daunomycin, or pokeweed antiviral proteins (PAP, PAPII, PAP-S), and examples of photo-activated agents include dihydropyridine- and omega-conotoxin. Suitable radioactive agents include ¹²⁵I, ¹¹¹In, ¹²³I, ³²P and others described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Other covalent and non-covalent modifications of antibodies are embraced herein, including agents, which are co-administered or administered after the antibody, for example, to induce inhibition or killing of cancer cells which contain the antibody.

An antibody, hK11, or sample may be immobilized on a carrier or solid support which is capable of immobilizing cells, antibodies etc. For example, the carrier or support may be nitrocellulose, or glass, polyacrylamides, gabbros, and magnetite. Examples of other suitable carriers are agarose, cellulose, dextran, Sephadex, Sepharose, liposomes, carboxymethyl cellulose polystyrene, filter paper, ion-exchange resin, plastic, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). An immobilized antibody may be prepared by reacting the material with a suitable insoluble carrier using known chemical or physical methods, for example, cyanogen bromide coupling.

Time-resolved fluorometry may be used to detect a signal. For example, the method described in Christopoulos T K and Diamandis E P, Anal. Chem. 1992:64:342-346 may be used with a conventional time-resolved fluorometer.

Therefore, in accordance with an embodiment of the invention, a method is provided wherein a hK11 antibody is labelled with an enzyme, a substrate for the enzyme is added wherein the substrate is selected so that the substrate, or a reaction product of the enzyme and substrate, forms fluorescent complexes with a lanthanide metal. A lanthanide metal is added and hK11 is quantitated in the sample by measuring fluorescence of the fluorescent complexes. The antibodies specific for hK11 may be directly or indirectly labelled with an enzyme. Enzymes are selected based on the ability of a substrate of the enzyme, or a reaction product of the enzyme and substrate, to complex with lanthanide metals such as europium and terbium. Examples of suitable enzymes include alkaline phosphatase and β-galactosidase. Preferably, the enzyme is alkaline phosphatase. The hK11 antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment the antibodies are biotinylated, and the enzyme is coupled to streptavidin.

In the method, antibody bound to hK11 in a sample is detected by adding a substrate for the enzyme.

The substrate is selected so that in the presence of a lanthanide metal (e.g. europium, terbium, samarium, and dysprosium, preferably europium and terbium), the substrate, or a reaction product of the enzyme and substrate, forms a fluorescent complex with the lanthanide metal. Examples of enzymes and substrates for enzymes that provide such fluorescent complexes are described in U.S. Pat. No. 5,3112,922 to Diamandis, and References 10, 25 and 30. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

In accordance with an embodiment, the present invention provides means for determining hK11 in a blood sample (e.g. serum) by measuring hK11 by immunoassay. It will be evident to a skilled artisan that a variety of immunoassay methods can be used to measure hK11. In general, an hK11 immunoassay method may be competitive or noncompetitive. Competitive methods typically employ an immobilized or immobilizable antibody to hK11 (anti-hK11) and a labeled form of hK11. Sample hK11 and labeled hK11 compete for binding to anti-hK11. After separation of the resulting labeled hK11 that has become bound to anti-hK11 (bound fraction) from that which has remained unbound (unbound fraction), the amount of the label in either bound or unbound fraction is measured and may be correlated with the amount of hK11 in the test sample in any conventional manner, e.g., by comparison to a standard curve.

Preferably a non-competitive method is used for the determination of hK11, with the most common method being the “sandwich” method. In this assay, two anti-hK11 antibodies are employed. One of the anti-hK11 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises hK11 bound by (“sandwiched” between) the capture and detection antibodies.

In a typical two-site immunometric assay for hK11, one or both of the capture and detection antibodies are polyclonal antibodies. The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody is selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against hK11 (e.g. an anti-hK11 monoclonal antibody and an anti-hK11 polyclonal antibody), a second antibody having specificity against an antibody reactive against hK11 labelled with an enzymatic label (e.g. enzyme conjugated to an antibody specific for the anti-hK11 polyclonal antibody), and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using conventional fluorimetry or a time-resolved fluorometer as described herein. In another embodiment, the enzyme is alkaline phosphatase (ALP) and the substrate is diflunisal phosphate (DIFP). ALP cleaves phosphate from DIFP to produce diflunisal (DIF) which forms a highly fluorescent terbium complex that can be monitored by time resolved or conventional fluorimetry.

Nucleic acid methods can also be used to detect transcription levels of hK11 as a marker of abnormal cell growth indicative of prostate or ovarian cancer. PCR and other nucleic acid methods, such as ligase chain reaction (LCR), and nucleic acid sequence based amplification (NASABA) can be used to detect malignant cells. For example, RT-PCR can be used to detect the presence of a specific mRNA population in a complex mixture of mRNA species.

The expression and quantitation of a hK11 gene can be detected using hybridization to clones arrayed on a grid (gridding). For example, a cDNA encoding a hK11 gene can be fixed to a substrate (e.g. glass, nitrocellulose, nylon, or plastic), and incubated with an analyte which maybe RNA or a cDNA copy of the RNA isolated from the tissue of interest. Hybridization between the substrate bound clone and the analyte can be detected and quantitated by several means including radioactive labeling or fluorescence labeling of the analyte or a secondary molecule that is designed to detect the hybrid. The quantitation of gene expression levels can be done by comparison of the intensity of the signal from the analyte compared with that determined from known standards. Standards may be obtained by in vitro transcription of the gene encoding hK11, quantitating the yield, and then using that material to generate a standard curve.

Antibodies specific for hK11 may be used in immunohistochemical analyses, for example, at the cellular and sub-subcellular level, to detect a hK11 protein, to localize it to particular ovarian or prostate tumor cells and tissues, and to specific subcellular locations, and to quantitate the level of expression.

Cytochemical techniques known in the art for localizing antigens using light and electron microscopy may be used to detect a hK11 protein. Generally, an antibody may be labeled with a detectable substance and an hK11 protein may be localised in tissues and cells based upon the presence of the detectable substance.

Where a radioactive label is used as a detectable substance, a hK11 protein may be localized by radioautography. The results of radioautography may be quantitated by determining the density of particles in the radioautographs by various optical methods, or by counting the grains.

In an aspect of the invention, antibodies specific for hK11 are used in imaging methodologies in the management of ovarian or prostate cancer. The invention provides a method for imaging tumors associated with one or more kallikreins, preferably kallikreins associated with ovarian cancer, most preferably hK11.

The invention also contemplates imaging methods described herein using multiple markers for ovarian or prostate cancer. For example, a method for imaging ovarian cancer may further comprise injecting the patient with one or more of an agent that binds to human stratum corneum chymotryptic enzyme (HSCCE), kallikrein 4, kallikrein 5, kallikrein 6, kallikrein 8, kallikrein 9, kallikrein 10, kallikrein 15, CA125, CA15-3, CA19-9, OVX1, lysophosphatidic acid (LPA) or carcinoembryonic antigen (CEA), preferably CA125. Preferably each agent is labeled so that it can be distinguished during the imaging. A method for imaging prostate cancer may further comprise injecting the patient with one or more of an agent that binds to kallikrein 2, kallikrein 5, kallikrein 10, kallikrein 15, HER-2, and prostate-specific antigen.

In an embodiment the method is an in vivo method and a subject or patient is administered one or more agents that carry an imaging label and are capable of targeting or binding to a kallikrein. The agent is allowed to incubate in vivo and bind to the kallikrein(s) associated with a tumor, preferably ovarian or prostate tumors. The presence of the label is localized to the ovarian or prostate cancer, and the localized label is detected using imaging devices known to those skilled in the art.

The agent may be an antibody or chemical entity which recognizes the kallikrein(s). In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the kallikreins used in the methods of the invention may be obtained from scientific or commercial sources, or isolated native kallikrein or recombinant kallikrein may be utilized to prepare antibodies etc. as described herein.

An agent may be a peptide that mimics the epitope for an antibody specific for a kallikrein and binds to the kallikrein. The peptide may be produced on a commercial synthesizer using conventional solid phase chemistry. By way of example, a peptide may be prepared that includes either tyrosine lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a kallilkrein producing tumor.

The agent carries a label to image the kallikreins. The agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, 47Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{119m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature ( see Hunter and Greenwood, Nature 144:945 (1962), David et al., Biochemistry 13:1014-1021(1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry ,289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

In the case of a radiolabeled agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example A. R. Bradwell et al.], “Developments in Antibody Imaging”, Monoclonal Antibodies for Cancer Detection and Therapy, R. W. Baldwin et al., (eds.), pp. 65-85 (Academic Press 1985)). A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. For ovarian or prostate cancer, administration preferably is intravenous. The bio-distribution of the label can be monitored by scintigraphy, and accumulations of the label are related to the presence of cancer cells. Whole body imaging techniques are described in U.S. Pat. Nos. 4,036,945 and 4,311,688. Other examples of agents useful for diagnosis and therapeutic use which can be coupled to antibodies and antibody fragments include metallothionein and fragments (see, U.S. Pat. No. 4,732,864). These agents are useful in diagnosis staging and visualization of cancer, in particular ovarian or prostate cancer, so that surgical and/or radiation treatment protocols can be used more efficiently.

The invention also contemplates kits for carrying out the methods of the invention. The kits may include an antibody or an antibody fragment which binds specifically to an epitope of a kallikrein, and means for detecting binding of the antibody to its epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

The diagnostic methods of the invention can be carried out using a diagnostic kit for quantitating hK11 in a sample. By way of example, the kit may contain antibodies specific for hK11, antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

The invention also provides methods using nucleic acid molecules to suppress the growth of a hK11 expressing cancer cell.

Genes encoding a hK11 protein can be turned off by transfecting a cell or tissue with vectors which express high levels of a desired hK11-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA. Antisense nucleic acid molecules may be to the regulatory regions of a gene encoding a hK11 protein, i.e., the promoters, enhancers, and introns. Preferably, oligonucleotides are derived from the transcription initiation site, e.g., between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a hK11 protein.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods known to the skilled artisan which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Still further the invention provides a method for evaluating a compound for its ability to modulate the biological activity of a hK11 protein and thus is potentially useful in treating prostate or ovarian cancer. For example, a substance which binds to hK11 or inhibits or enhances the interaction of the protein and a substance which binds to the protein may be evaluated. In an embodiment, the method comprises providing a known concentration of a hK11 protein, with a substance which binds to the protein and a test compound under conditions which permit the formation of complexes between the substance and protein, and removing and/or detecting complexes.

In other embodiments, the invention provides a method for identifying inhibitors of a hK11 protein comprising:

(a) providing a reaction mixture including the hK11 protein and a substance that binds to the hK11 protein, or at least a portion of each which interact;

(b) contacting the reaction mixture with one or more test compounds;

(c) identifying compounds which inhibit the interaction of the hK11 protein and substance.

Compounds which modulate the biological activity of a hK11 protein may also be identified by comparing the pattern and level of expression of the protein in tissues and cells, in the presence, and in the absence of the compounds. In addition, compounds that modulate the biological activity of a hK11 protein may be identified by assaying for modulation (i.e. inhibition or enhancement) of enzymatic activity.

In certain preferred embodiments, the reaction mixture used in a method of the invention is a whole cell. In other embodiments, the reaction mixture is a cell lysate or purified protein composition. The subject methods can be carried out using libraries of test compounds. Such agents can be proteins, peptides, nucleic acids, carbohydrates, small organic molecules, and natural product extract libraries, such as those isolated from animals, plants, fungus and/or microbes.

Still another aspect of the present invention provides a method of conducting a drug discovery business comprising:

(a) providing one or more assay systems for identifying agents by their ability to bind to or inhibit or potentiate the interaction of a hK11 protein and a substance that binds to the protein, or by their ability to modulate the enzymatic activity of a hK11 protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The antibodies, antisense nucleic acid molecules, and substances and compounds identified in accordance with the invention, may be used to modulate the biological activity of a hK11 protein, and they may be used in the treatment of conditions such as prostate and ovarian cancer. Accordingly, the substances and compounds may be formulated into compositions for administration to individuals suffering from disorders such as prostate and ovarian cancer. In particular, the antibodies, antisense nucleic acid molecules, substances and compounds may be used to treat patients who have a hK11 protein in, or on, their cancer cells.

Therefore, the present invention also relates to a composition comprising one or more of a substance or compound identified using the methods of the invention, and a pharmaceutically acceptable carrier, excipient or diluent. A method for treating or preventing a disorder such as cancer is also provided comprising administering to a patient in need thereof, a substance or compound identified using the methods of the invention, antibody, hK11 antisense molecules, or a composition of the invention.

The invention also provides immunotherapeutic approaches for preventing or reducing the severity of a cancer (e.g. prostate or ovarian cancer). The clinical signs or symptoms of the cancer in a subject are indicative of a beneficial effect to the patient due to the stimulation of the subject's immune response against the cancer. Stimulating an immune response refers to inducing an immune response or enhancing the activity of immunoeffector cells in response. to administration of a vaccine preparation of the invention. The prevention of a cancer can be indicated by an increased time before the appearance of cancer in a patient that is predisposed to developing cancer due for example to a genetic disposition or exposure to a carcinogenic agent. The reduction in the severity of a cancer can be indicated by a decrease in size or growth rate of a tumor.

The invention broadly contemplates vaccines for stimulating or enhancing in a subject to whom the vaccine is administered production of antibodies directed against a hK11 protein.

The invention also provides a method for stimulating or enhancing in a subject production of antibodies directed against an hK11 protein. The method comprises administering to the subject a vaccine of the invention in a dose effective for stimulating or enhancing production of the antibodies.

The invention further provides methods for treating, preventing, or delaying recurrence of cancer, in particular prostate and ovarian cancer. The methods comprise administering to the subject a vaccine of the invention in a dose effective for treating, preventing, or delaying recurrence of the cancer.

Vaccines can be derived from a hK11 protein, peptides derived therefrom, or chemically produced synthetic peptides, or any combination of these molecules, or fusion proteins or peptides thereof. The proteins, peptides, etc. can be synthesized or prepared recombinantly or otherwise biologically, to comprise one or more amino acid sequences corresponding to one or more epitopes of a hK11 protein. Epitopes of a hK11 protein will be understood to include the possibility that in some instances amino acid sequence variations of a naturally occurring protein or polypeptide may be antigenic and confer protective immunity against cancer or anti-tumorigenic effects. Sequence variations may include without limitation, amino acid substitutions, extensions, deletions, truncations, interpolations, and combinations thereof. Such variations fall within the scope of the invention provided the protein containing them is immunogenic and antibodies against such polypeptide cross-react with naturally occurring hK11 protein to a sufficient extent to provide protective immunity and/or anti-tumorigenic activity when administered as a vaccine.

The hK11 proteins, peptides etc, can be incorporated into vaccines capable of inducing an immune response using methods known in the art. Techniques for enhancing the antigenicity of the proteins, peptides, etc. are known in the art and include incorporation into a multimeric structure, binding to a highly immunogenic protein carrier, for example, keyhole limpet hemocyanin (KLH), or diptheria toxoid, and administration in combination with adjuvants or any other enhancer of immune response.

Vaccines may be combined with physiologically acceptable media, including immunologically acceptable diluents and carriers as well as commonly employed adjuvants such as Freund's Complete Adjuvant, saponin, alum, and the like.

It will be further appreciated that anti-idiotype antibodies to antibodies to hK11 protein are also useful as vaccines and can be similarly formulated.

The administration of a vaccine is generally applicable to the prevention or treatment of cancer, in particular prostate and ovarian cancer.

The administration to a patient of a vaccine in accordance with the invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. The cancer immunotherapy in accordance with the invention would be a preferred treatment for the prevention and/or treatment of cancer, since the side effects involved are substantially minimal compared with the other available treatments e.g. surgery, chemotherapy, radiation therapy. The vaccines have the potential or capability to prevent cancer (in particular prostate and ovarian cancer) in subjects without cancer but who are at risk of developing cancer.

The following non-limiting example is illustrative of the present invention:

### EXAMPLE 1

### Materials and Methods

Recombinant hK11 protein was produced using the procedures described in detail elsewhere for hK10 protein (11). Briefly, the complete KLK11 cDNA coding sequence was cloned into the EasySelect™ Pichia pastoris yeast expression system (Invitrogen). The accuracy of the sequence of the insert was verified by double-stranded DNA sequencing. A stable yeast clone was identified and cultured in the presence of methanol for five days. The cells were then spun down and the supernatant, containing the recombinant hK11, was collected.

The recombinant hK11 protein was purified from the supernatant by using cation-exchange chromatography followed by reverse-phase gradient chromatography on a Vydac C4 column, as described (11). The presence of hK11 in fractions was verified with Western blotting, using an anti-hK11 peptide antibody. The purity and molecular mass of purified recombinant hK11 was assessed by sodium dodecyl sulphate-polyacrylamide gel electrophoresis and staining with Commassie Blue. The protein concentration of the purified recombinant hK11 was determined by the bicinchoninic acid method with bovine serum albumin as a calibrator (Pierce Chemical Co.). Positive identification and characterization of the recombinant hK11 protein was achieved by using trypsin digestion and nanoelectrospray mass spectrometry, as previously described (11).

Production of Polyclonal and Monoclonal Antibodies Against hK11:

The purified recombinant hK11 protein was used to immunize rabbits and mice. hK11 (100 μg) was injected subcutaneously into female Balb/c mice and New Zealand white rabbits. The protein was diluted in complete Freund's adjuvant for the first injection, and in incomplete Freund's adjuvant for subsequent injections. Injections were repeated six times at 3-week intervals for the rabbits and three times for the mice. Blood was drawn from the animals and tested for antibody generation. The screening strategies were similar to those described elsewhere for hK10 (11). The rabbit polyclonal antibodies were used for developing the immunofluorometric assay without further purification.

Monoclonal antibodies against hK11 were produced by using standard hybridoma technology, as described previously (38). Positive clones were identified by screening tissue culture supernatants, as described (11). The positive clones were expanded sequentially in 24-well plates and 6-well plates. Supernatants were further characterized by performing IgG isotyping and clones were subjected to limiting dilutions. The clones were then expanded in flasks to generate large amounts of supernatants in serum-free media. The monoclonal antibodies were purified from the supernatants by using protein G-affinity chromatography.

Immunofluorometric Assay for hK11

Standard Assay Procedure:

One purified anti-hK11 monoclonal antibody diluted in coating buffer (containing 50 mmol/L Tris, pH 7.80) was dispensed into a 96-well white polystyrene microtiter plate (100 μL/500 ng/well) and incubated overnight at room temperature. The plates were then washed three times with washing buffer (containing 9 g/L NaCl and 0.5 g/L Tween-20 in 10 mmol/L Tris-buffer, pH 7.8). One hundred μL of hK11 calibrators (recombinant hk11 in 60 g/L BSA) or samples were applied into each well along with 50 μL of assay buffer. The assay buffer was a 50 mmol/L Tris-buffer, pH 7.80, containing per liter 60 g of BSA, 0.5 mol KCl, 0.5 g Tween-20, 10 g bovine immunoglobulins, 100 mL goat serum and 25 mL mouse serum. The plates were incubated for 2 h on an orbital shaker to allow for hK11 molecules to bind to the plates. The plates were then washed six times. Subsequently, the plates were incubated for 1 h with 100 μL per well of a rabbit anti-hK11 polyclonal antibody, diluted 2,000-fold in assay buffer. The plates were then washed six times with washing buffer. One hundred μL of alkaline phosphatase-conjugated goat anti-rabbit antibody (Jackson Immuno Research), diluted 3,000-fold in assay buffer were added to each well, incubated for 30 min and washed six times, as described above. Finally, 100 μL of 1 mmol/L diflunisal phosphate (DFP), diluted in substrate buffer(0.1 mol/L Tris, pH 9.1, 0.1 M NaCl and 1 mmol/L MgCl₂) were added into each well and incubated for 10 min. One hundred μL of developing solution (1 mol/L Tris-base, 0.4 mol/L sodium hydroxide, 2 mmol/L TbCl₃ and 3 mmol/L EDTA) were pipetted into each well and mixed for 1 min. The fluorescence was measured with a time-resolved fluorometer on the CyberFluor 615 Immunoanalyzer (MDS Nordion, Kanata, Ontario, Canada), as previously described (10). The calibration and data reduction was performed automatically.

Determination of Assay Characteristics.

The assay characteristics were determined essentially as described elsewhere ( 11).

Human Tissue Cytosolic Extracts and Biological Fluids.

Human tissue cytosolic extracts were prepared as follows. Various frozen human tissues (0.2 g) were pulverized on dry ice to fine powders. Extraction buffer (1 mL, containing 50 mmol/L Tris, pH 8.0, 150 mmol/L NaCl, 5 mmol/L EDTA, 10 g/L NP-40 surfactant, 1 mmol/L phenylmethylsulfonlyfluoride, 1 g/L aprotinin, 1 g/L leupeptin) was added to the tissue powders and the mixture was incubated on ice for 30 min with repeated shaking and mixing every 10 min. Mixtures were then centrifuged at 14,000 rpm at 4° C. for 30 min. The supernatants (cytosolic extracts) were then collected. The biological fluids tested were leftovers of samples submitted for routine biochemical testing. All tissue cytosolic extracts and biological fluids were stored at −80° C. until use.

Recovery.

Recombinant hK11 was added to human serum samples at different concentrations and measured with the developed hK11 immunoassay. Recoveries were then calculated after subtraction of the endogenous concentrations.

Fractionation of Biological Fluids with Size-Exclusion HPLC.

Biological fluids were fractionated on a silica-based gel filtration column essentially as described elsewhere (11). The fractions were collected and analyzed for hK11 with the developed immunoassay.

Immunohistochemistry.

A mouse monoclonal antibody was raised against hK11 full-size recombinant protein produced in yeast cells, as described above. Immunohistochemical staining for hK11 was performed according to a standard immunoperoxidase method. Briefly, paraffin-embedded tissue sections (4 μm) were fixed and dewaxed. Endogenous peroxidase activity was blocked with 3% aqueous hydrogen peroxide for 15 min. Sections were then treated with 0.4% pepsin at pH 2.0for 5 min at 42° C. and blocked with 20% protein blocker (Signet Labs) for 10 min. The primary antibody was then added at 1:3,000 dilution for 1 h at room temperature. After washing, biotinylated anti-mouse antibody (Signet Labs) was added, diluted 4-fold in antibody dilution buffer (Dako). Following incubation and washing, streptavidin-tagged horseradish peroxidase was added for 30 min at room temperature. After washing, detection was achieved with amino ethyl carbazol (AEC) for 5-10 min. The slides were then counter-stained with hematoxylin and then mounted with cover slips.

Hormonal Regulation of hK11 .

To study the hormonal regulation of hK11, various cell lines were cultured and then stimulated with different steroids, essentially as described elsewhere (39). All steroid hormones were used at the final concentration of 10⁻⁸ M. Tissue culture supernatants were collected after seven days and used for analysis of hK11 as well as of prostate-specific antigen, as a control. PSA analysis was performed with the method described elsewhere (40).

Results

Recombinant hK11 protein was produced in both bacterial and yeast expression systems. The protein was purified using successive cycles of ion-exchange chromatography and reverse-phase liquid chromatography, as previously described (11). The identity of the protein was verified by nanoelectro spray mass spectrometry (11). The concentration of the highly purified protein (>99% by SDS-PAGE) was established by the bicinchoninic acid total protein method with bovine serum albumin as standard.

Monoclonal mouse antibodies and polyclonal rabbit antibodies were raised using standard techniques (38). One high affinity monoclonal antibody was used for coating microtiter plates (capture antibody) and the polyclonal rabbit antibody was used for detection, in an ELISA-type sandwich assay. A secondary goat anti-rabbit polyclonal antibody, labeled with alkaline phosphatase, was also used and the activity of alkaline phosphatase was detected by time-resolved fluorometry, as previously described (10). This assay was carefully optimized in terms of amounts of reagents used and incubation times, in order to obtain the lowest possible detection limits. The optimal conditions are described above.

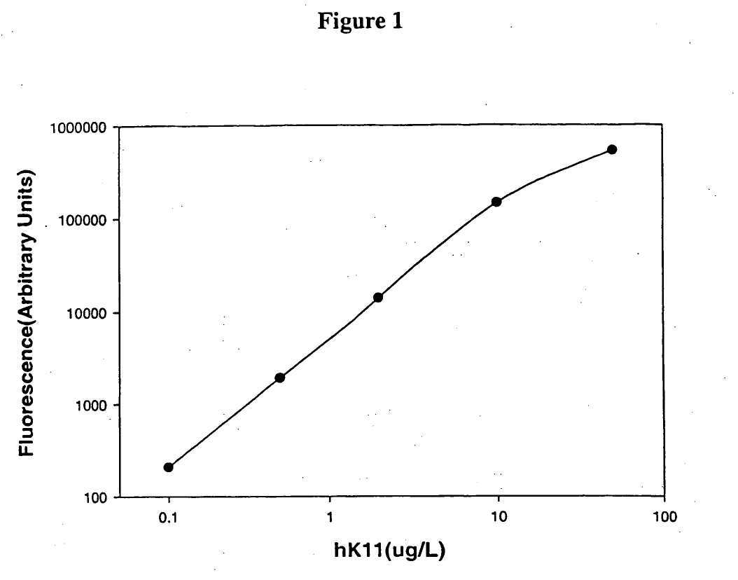

A typical calibration curve for this assay is shown in FIG. 1. The detection limit, defined as the concentration of analyte that can be distinguished from zero with 95% confidence, is 0.1 μg/L and the dynamic range extends to 50 μg/L. Within-run and day-to-day precision studies yielded CVs<10% within the measurement range. Recovery of added recombinant hK11 to serum averaged 50% (Table 1). The cross-reactivity of this assay was further evaluated against other homologous kallikreins. No detectable cross-reactivity was found from hK2, hK4, hK6, hK10 and hK13 when these recombinant proteins (produced in-house) were tested at levels up to 1,000 μg/L. For PSA (hK3), some cross-reactivity was detected when the PSA used was purified from seminal plasma. At 1,000 μg/L and 10,000 μg/L of seminal plasma PSA, the hK11 equivalent concentrations were 0.23 and 3.2 μg/L, respectively, yielding an average cross-reactivity of 0.028%. However, when the cross-reactivity was tested with recombinant PSA, produced in bacteria, the cross-reactivity was much less (0.0003%). Thus it was concluded that the higher cross-reactivity found with seminal plasma PSA, was likely due to hK11 contamination in this preparation. Further evidence that PSA does not cross-react with the hK11 immunoassay is provided in the following paragraphs.

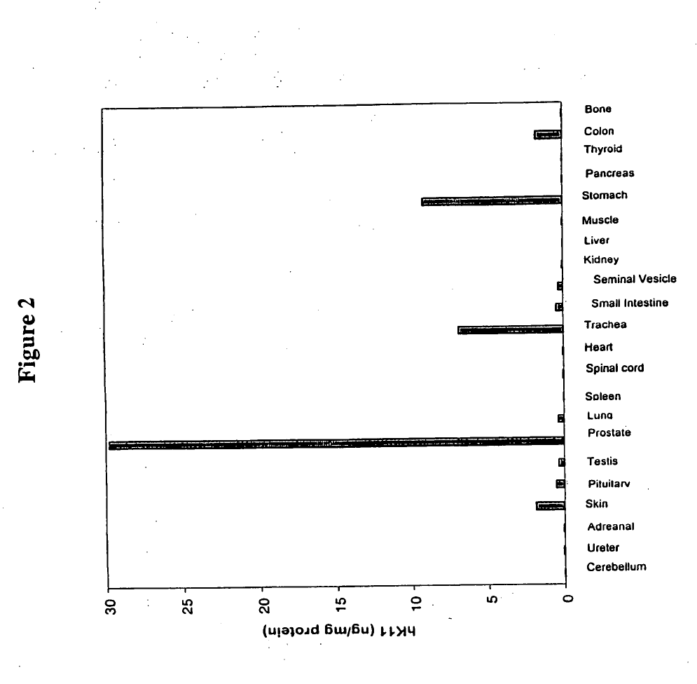

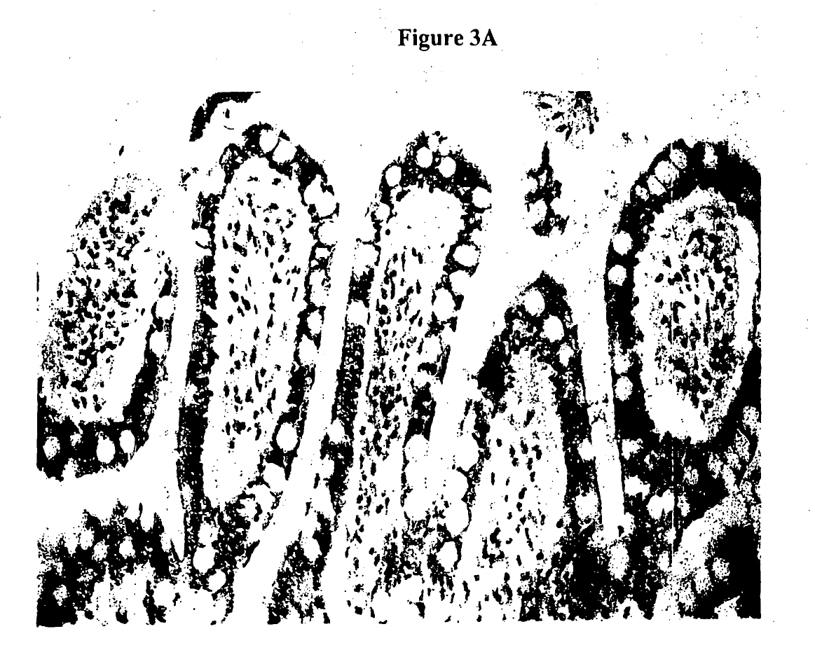

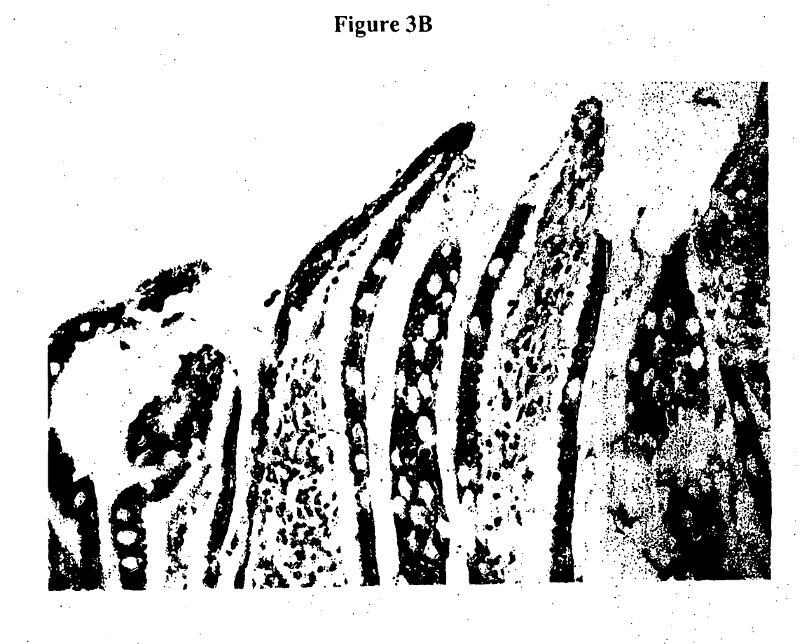

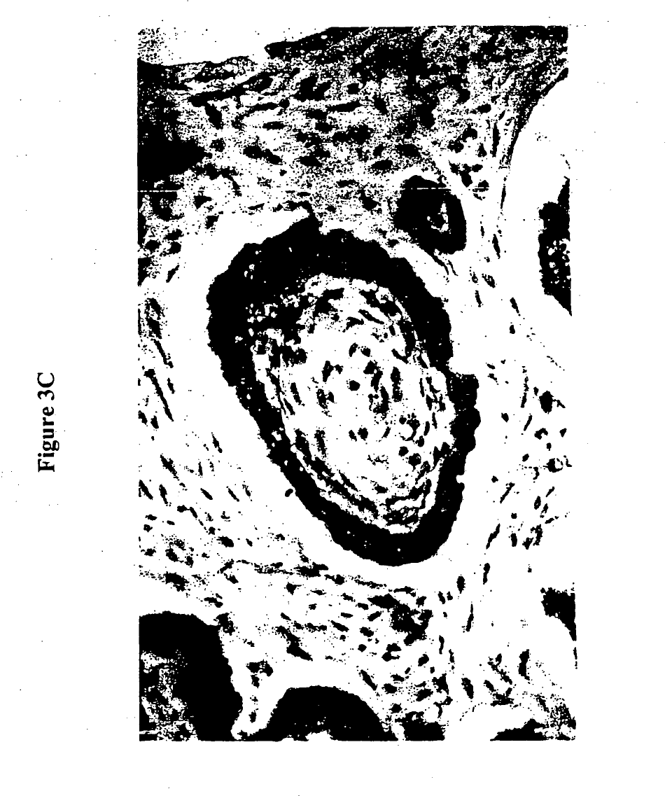

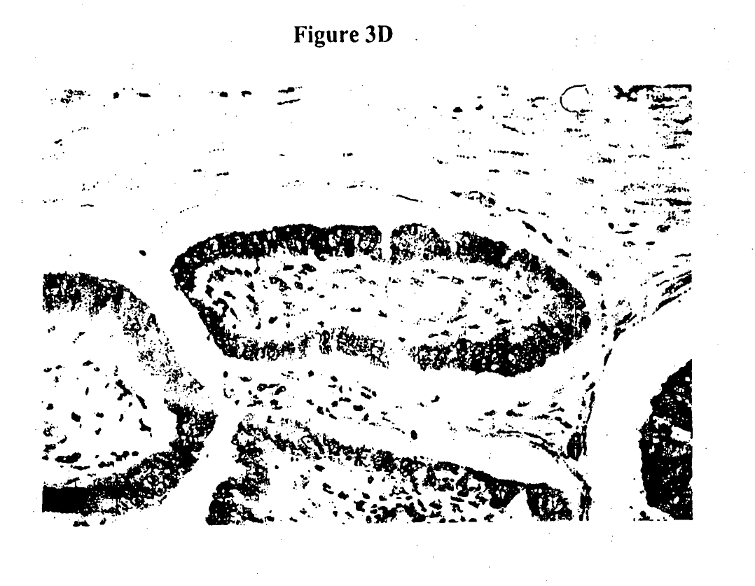

In order to study the distribution of hK11 protein in various human tissues, cytosolic extracts were prepared as described above, and hK11 was quantitated by the developed immunoassay. All values were corrected for the total protein content of the extracts. The data are shown in FIG. 2. Highest levels of hK11 were found in the prostate, followed by stomach, trachea, skin and colon. Lower levels were found in pituitary, testis, lung, small intestine and seminal vesicles. In order to study the cellular distribution of hK11, it was immunohistochemically localized in paraffin-embedded tissue in intestinal epithelium (FIGS. 3A, B). Staining was restricted to the cytoplasm of epithelial cells.

The presence of hK11 was further tested in various biological fluids . The data are shown in Table 2. hK11 was detected in all fluids tested, with highest levels seen in amniotic fluid and milk of lactating women. The presence of hK11 in these fluids provides further evidence that hK11 is a secreted protein.

hK11 was further quantitated in seminal plasma, given the fact that this serine protease is present at highest levels in the prostate gland (FIG. 2). hK11 was also quantitated in five different prostatic tissue extracts along with PSA (hK3), to establish their relative abundances. These data are shown in Table 3. hK11 concentration in seminal plasma is, on average, more than 100-fold higher than in any other biological fluid shown in Table 2. In the prostatic tissue extracts, the levels of hK11 are approximately 250 times lower than PSA levels and there is no apparent correlation between PSA and hK11 levels. In seminal plasma, the levels of hK11 are also approximately 300 times lower than PSA. The lack of correlation between PSA and hK11 concentration in seminal plasma (Table 3) further suggests that the assay is not affected by the relatively huge amounts of PSA in this fluid. Another prostatic kallikrein, hK2, is present in seminal plasma at levels approximately 100-500 times lower than PSA (41). Thus, hK11 appears to be present in seminal plasma at levels 300 times lower than PSA and at levels roughly equivalent to those of hK2.

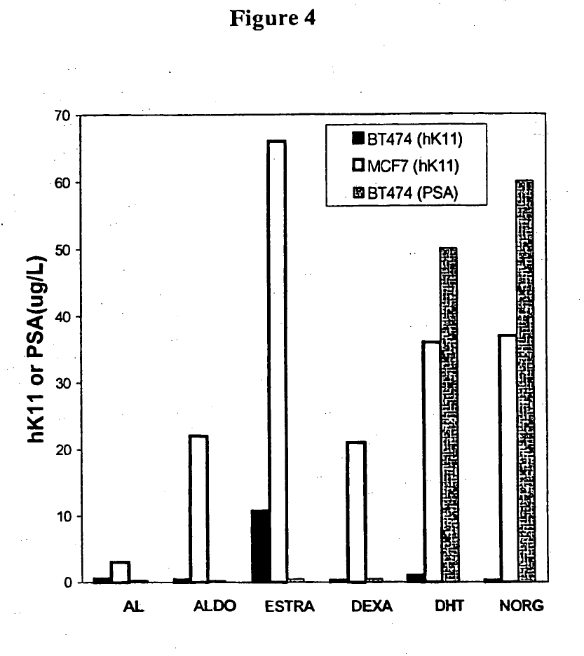

Tissue culture systems have been previously established for studying the hormonal regulation of hK3 and hK2 (39). The cell lines used in this study were LNCaP (prostatic carcinoma), PC-3 (AR) (a prostatic carcinoma cell line stably transfected with androgen receptor), MCF-7 (breast carcinoma), MFM-223 (breast carcinoma), ZR-75 (breast carcinoma) BT-474 (breast carcinoma), T-47D (breast carcinoma), BT20 (breast carcinoma) and BG-1 (ovarian carcinoma). From all these cell lines, only two (MCF-7 and BT-474) were able to produce and secrete detectable hK11 upon hormonal stimulation. As can be seen from FIG. 4, hK11 protein production is highly stimulated mainly by estradiol in both cell lines, and to a lower degree, by other steroid hormones. In contrast, PSA (hK3) is highly up-regulated by dihydrotestosterone (DHT) and the progestin norgestrel in BT-474 cells. In accordance with previous data, no production of PSA was found in the MCF-7 cell line (39). These data strongly suggest that the KLK11 gene is up-regulated mainly by estrogens in these cell lines.

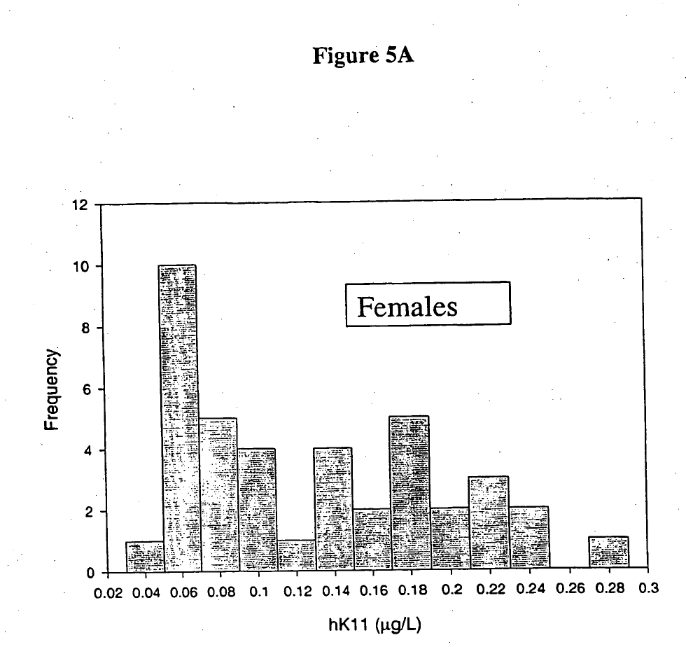

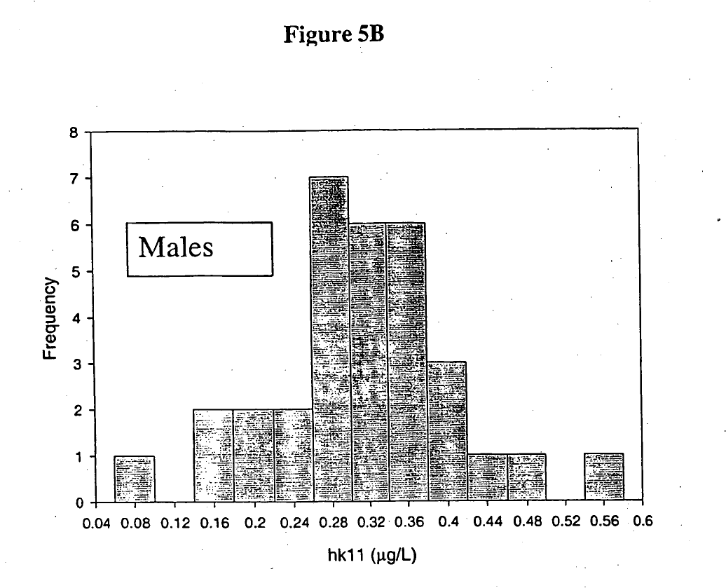

hK11 was quantitated in serum of 40 apparently healthy females (ages 25 to 60) and 32 males (ages 30 to 65). The distribution of obtained values is shown in FIGS. 5A and 5B. Males have approximately three times higher levels, apparently due to the presence of the prostate. The medians of males (0.32 μg/L) and females (0.11 μg/L) differ significantly (p<0.01 by Mann-Whitney test). For further analyses, an upper reference range of 0.25 μg/L for women and 0.50 μg/L for men (95^{th} percentile) were considered.

A total of 114 serum samples from patients with various malignancies were analyzed to examine if hK11 is elevated in any of them. The data are presented in Table 4. Highest percentages of hK11 elevations were seen in patients with ovarian and prostate cancers. The immunohistochemical localization of hK11 in an ovarian cancer tissue is shown in FIGS. 3C, D.

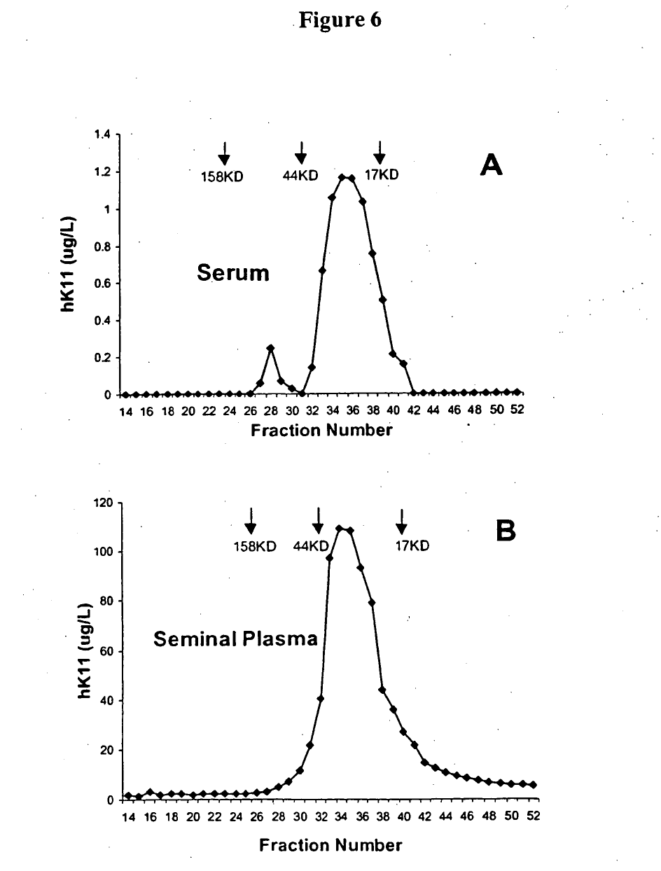

In order to examine the molecular forms of hK11 inseminal plasma and serum, as determined by the assay, one seminal plasma and one serum sample with high hK11 were separated on a gel filtration column. The data are shown in FIG. 6. hK11 in seminal plasma elutes as a single peak corresponding to a molecular weight of 30 kDa (free hK11). In serum, in addition to the major 30 kDa form, there is a small peak (<10%) corresponding to a molecular mass of approximately 100 kDa. This may represent hK11 bound to serum proteinase inhibitors, as has been shown for PSA (42,43).

Discussion

Among all known cancer biomarkers, prostate-specific antigen (PSA) is the most valuable, due to its tissue specificity. Elevated serum PSA levels are seen in patients with prostatic carcinoma and the test is widely used for diagnosis and monitoring of this disease. PSA is a member of the human tissue kallikrein gene family and it is a secreted serine protease of 30 kDa molecular mass (5, 6, 25, and 44). Another member of this family, human glandular kallikrein 2 (hK2), is an emerging biomarker for prostatic carcinoma (5). More recently, two other members of the kallikrein gene family, hK6 and hK10, have been proposed as new biomarkers for ovarian carcinoma (26, 27). Although there are now 15 known kallikrein genes, many family members have not been studied in detail (6, 25).

Recombinant hK11 protein was produced in both yeast and bacterial expression systems. These proteins were highly purified by chromatography and used as immunogens to produce monoclonal and polyclonal antibodies. These antibodies were used to develop a highly sensitive immunoassay which is suitable for hK11 quantification in biological fluids and tissue extracts. Like a few other kallikreins (hK2, hK3, hK4, hK6, hK10) (6, 25), hK11 is highly expressed in the prostate gland and to a lower degree in a number of other tissues (FIG. 2). Many biological fluids, including amniotic fluid and milk of lactating women contain considerable amounts of hK11 (Table 2). However, highest levels are seen in seminal plasma (Table 3).

hK11 is secreted by epithelial cells and has been immunolocalized in the supranuclear compartment, likely representing the Golgi apparatus of these cells (FIG. 3). Like many other members of this family, hK11 is up-regulated by steroid hormones, and especially estradiol, in two breast cancer cell lines (FIG. 4). Other members of this family (e.g. PSA, hK2 and hK4) are up-regulated by androgens (6, 25, 39) while other kallikreins are up-regulated by estrogens (6, 25, 45).

The assay described herein detects mainly the free form of hK11 in biological fluids (FIG. 6). The data with serum suggest that hK11 may also be present in a complexed form with proteinase inhibitors, like other kallikreins (42, 43). The lower recovery in serum (−50%; Table 1) further suggests that hK11 may bind to proteinase inhibitors upon entrance into the circulation.

Although hK11 is present in the prostate, its concentration is substantially lower than PSA (Table 3).

However, it appears that hK11 and hK2 concentrations are approximately equal in both prostatic tissue extracts and seminal plasma (41). It has previously been shown that PSA can cleave semenogelins and facilitate semen liquifaction (46). Additionally, hK2 can activate the pro-form of PSA (47-49). More recently, hK15, another kallikrein expressed in the prostate (33), was shown to activate the pro-form of PSA more efficiently than hK2 (50).

Serum hK11 concentration is elevated in the majority of patients with ovarian and prostate cancer (Table 4). hK11 protein was immunohistochemically localized in ovarian cancer tissue (FIG. 3). Positivity was predominantly seen in the cytoplasm of tumor cells.

The present invention is not to be limited in scope by the specific embodiments described herein, since such embodiments arc intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

Below full citations are set out for the references referred to in the specification.

TABLE 1
Recovery of added recombinant hK11 to human serum

| | hK11, μg/L | | | |
|---|---|---|---|---|
| | Initially | | | |
| Sample | Present | Added | Recovered | % Recovery |
| 60 g/L bovine serum | 0 | 4.9 | 4.9 | 100 |
| albumin | | 16.9 | 16.9 | 100 |
| (control) | | | | |
| Male serum #1 | 0.27 | 4.9 | 2.2 | 46 |
| | | 16.9 | 8.6 | 51 |
| Males serum #2 | 0.17 | 4.9 | 2.7 | 55 |
| | | 16.9 | 9.2 | 55 |
| Female serum #1 | 0.24 | 4.9 | 2.4 | 48 |
| | | 16.9 | 8.0 | 47 |
| Female serum #2 | 0.23 | 4.9 | 2.4 | 48 |
| | | 16.9 | 8.6 | 51 |
| | | | Average: | 50 |

TABLE 2
Concentration of hK11 in biological fluids

| | hK11, μg/L | | | | | |
|---|---|---|---|---|---|---|
| | Sample # | | | | | |
| Fluid | 1 | 2 | 3 | 4 | 5 | 6 |
| Milk of lactating women | 0.8 | 40 | 1.6 | 9.4 | 0.7 | 8.1 |
| Amniotic fluid | 5.2 | 45 | 2.0 | 7.8 | 10.5 | 11.3 |
| Cerebrospinal fluid | 2.9 | 3.0 | 1.5 | 1.6 | 1.3 | 1.7 |
| Follicular fluid | 0.5 | 2.7 | 0.7 | 7.3 | 0.6 | 2.2 |
| Breast cancer cytosol | 0.16 | 0.13 | <0.1 | 0.31 | 0.1 | 0.8 |

TABLE 3
Concentrations of PSA (hK3) and hK11 in prostatic
tissue extracts and seminal plasmas

| | PSA, μg/L | hK11, μg/L |
|---|---|---|
| Prostatic Tissue | | |
| Extracts | | |
| 1 | 69,000 | 766 |
| 2 | 11,000 | 192 |
| 3 | 12,000 | 146 |
| 4 | 57,000 | 204 |
| 5 | 192,000 | 428 |
| Seminal Plasmas | | |
| w′1 | 3.5 × 10⁶ | 2.8 × 10³ |
| 2 | 1.1 × 10⁶ | 1.7 × 10³ |
| 3 | 0.3 × 10⁶ | 1.4 × 10³ |
| 4 | 0.9 × 10⁶ | 3.0 × 10³ |
| 5 | 0.6 × 10⁶ | 3.4 × 10³ |

TABLE 4
Concentration of hK11 in serum of patients with
various malignancies

| | Number of | Patients (%) | Patients (%) |
|---|---|---|---|
| | Serum | with hK11 > | with hK11 > |
| Malignancy | Samples | 0.50 μg/L^{1,2} | 1 μg/L |
| Prostate cancer | 20 | 12 (60%) | 6 (30%) |
| Ovarian cancer | 20 | 10 (50%) | 8 (40%) |
| Medullary thyroid | 15 | 5 (33%) | 1 (7%) |
| carcinoma | | | |
| Colon cancer | 28 | 6 (21%) | 2 (7%) |
| Lung cancer | 20 | 0 (0%) | 0 (0%) |
| Pancreas | 11 | 0 (0%) | 0 (0%) |

¹This value represents the 95^{th} percentile of healthy males.
²For women with ovarian cancer, 70% of them had hK11 above 0.25 μg/L (95^{th} percentile of healthy women).

Full Citations for References Referred to in the Specification

1. Bhoola K, Figueroa C and Worthy K. Bioregulation of kinins: kallikreins, kininogens, and kininases. Pharmacol Rev 1992; 44:1-80. [Review].

2. Clements J A. The molecular biology of the kallikreins and their roles in inflammation. In: Farmer S G (ed) The Kinin System, Academic Press, 1997; 5, 71-97. [Review].

3. Riegman P H, Vlietstra R J, Suurmeijer L, Cleutjens C B and Trapman J. Characterization of the human kallikrein locus. Genomics 1992; 14:6-11.

4. McCormack R T, Rittenhouse H G, Finlay J A, Sokoloff R L, Wang T J, Wolfert R L, Lilja H and Oesterling J E. Molecular forms of prostate-specific antigen and the human kallikrein gene family: a new era. Urology 1995; 45:72944. [Review].

5. Rittenhouse H G, Finlay J A, Mikolajczyk S D and Partin A W. Human Kallikrein 2 (hK2) and prostate-specific antigen (PSA): two closely related, but distinct, kallikreins in the prostate. Crit Rev Clin Lab Sci 1998; 35:275-368. [Review].

6. Yousef G M, Diamandis E P. The new human tissue kallikrein gene family: Structure, function and association to disease. Endocr Rev 2001; 22:184-204.

7. Diamandis E P, Yousef G, Clements J, Ashworth L, Yoshida S, Egelrud T, Nelson P, Shiosaka S, Little S, Lilja H, Stenman U-H, Rittenhouse H and Wain H. New nomenclature for the human tissue kallikrein gene family. Clin Chem 2000;46:1855-1858.

8. Yoshida S, Taniguchi M, Suemoto T, Oka T, He X, Shiosaka S. cDNA cloning and expression of 1 novel serine protease, TLSP. Biochim Biophys Acta 1998;1399:225-228.

9. Yousef G M, Scorilas A, Diamandis E P. Genomic organization, mapping tissue expression and hormonal regulation of trypsin-like serine protease (TLSP PRSS20), a new member of the human kallikrein gene family. Genomics 2000;63:88-96.

10. Christopoulos T K, Diamandis E P. Enzymatically amplified time-resolved fluorescence immunoassay with terbium chelates. Anal Chem 1992;64:342-346.

11. Luo L Y, Grass L, Howarth D J C, Thibault P, Ong H and Diamandis E P. Immunofluorometric assay of human kallikrein 10 (hK10; NES1) and its identification in biological fluids and tissues. Clin Chem 2001;47:237-246.

12. Diamandis E P. Prostate specific antigen—its usefulness in clinical medicine. Trends Endocrinol Metab 1999; 25: 14-16.

13. McCormack R T, Rittenhouse H G, Finlay J A, Sokoloff R L, Wang T J, Wolfert R L, Lilja H and Oesterling J E. Molecular forms of prostate-specific antigen and the human kallikrein gene family: a new era. Urology 1995; 45:729-44. [Review].

14. Chu T M. Prostate-specific antigen and early detection of prostate cancer. Tumor Biol 1997; 18:123-134.

15. Stenman U-11. New ultrasensitive assays facilitate studies on the role of human glandular kallikrein (hK2) as a marker for prostatic disease. Clin Chem 1999;45:753-754.

16. Riman T, Persson I, Staffan N. Hormonal aspects of epithelial ovarian cancer: review of epidemiological evidence. Clin Endocrinol 1998;49:695-707.

17. National Cancer Institute of Canada: Canadian Cancer Statistics 1999, Toronto, Canada, 1999.

18. Rosenthal A N, Jacobs I J, The role of CA 125 in screening for ovarian cancer. Int J Biol Markers 1998;13:216-220.

19. Maggino T, Gadducci A. Serum markers as prognostic factors in epithelial ovarian cancer: an overview. Eur J Gynaecol Oncol 2000;21:64-69.

20. Bast R C Jr, Xu F J, Yu Y H, Barnhill S, Zhang Z, Mills G B, CA 125: the past and the future. Int J Biol Markers 1998;13:179-187.

21. Berek J S, Bast R C Jr. Ovarian cancer screening. The use of serial complementary tumor markers to improve sensitivity and specificity for early detection. Cancer 1995;76:2092-2096.

22. Lambert-Messerlian G M. Is inhibin a serum marker for ovarian cancer? Eur J Endocrinol 2000,42:331-333.

23. Burger H G, Baillic A, Drummond A E, Healy D L, Jobling T, Manners P, Robertson D M, et al. Inhibin and ovarian cancer. J Reprod Immunol 1998;39:77-87.

24. Xu F J, Yu Y H, Daly L, DeSombre K, Anselmino L, Hass G M, Berchuck A, et al. OVX1 radioimmunoassay complements CA-125 for predicting presence of residual ovarian carcinoma at second-look surgical surveillance procedures. J Clin Oncol 1993;11:1506-1510.

25. Diamandis E P, Yousef G M, Luo L Y, Magkiara A, Obiezu C V. The human kallikrein gene family—implications in carcinogenesis. Trends Endocrinol Metab 2000;11:54-60.

26. Diamandis E P, Yousef G M, Soosaipillai A R, Bunting P. Human kallikrein 6 (zyme/proteaseM/neurosin): A new serum biomarker of ovarian carcinoma. Clin Biochem 2000;33:579-583.

27. Luo L-Y, Bunting P, Scorilas A, Diamandis E P. Human kallikrein 10: a novel tumor marker for ovarian carcinoma? Clin Chim Acta 2001;306: 11-118.

28. Obiezu C V, Katsaros D, Massobrio M, Scorilas A, Yousef G M, Fracchioli S, Rigault de la Longrais I A, Arisio R, Diamandis E P. Higher human kallikrein gene 4 (KLK4) expression indicates poor prognosis of ovarian cancer patients. Clin Cancer Res. 2001; 7:2380-2386.

29. Kim H, Scorilas A, Katsaros D, Yousef G M, Massobrio M, Fracchioli S, Piccinno R, Gordini G, Diamandis E P. Human kallikrein gene 5 (KLK5) expression is an indicator of poor prognosis in ovarian cancer. Br J Cancer 2001 ;84:643-650.

30. Hoffman B R, Katsaros D, Scorilas A, Diamandis P, Fracchioli S, Rigault de la Longrais I A, Colgan T, Puopolo M, Giardina G, Massobrio M, Diamandis E P. Immuno-fluorometric quantitation and histochemical localization of kallikrein 6 protein in ovarian cancer tissue: a new independent unfavorable prognostic biomarker.

31. Magklara A, Scorilas A, Katsaros D, Massobrio M, Yousef G M, Fracchioli S, Danese S, Diamandis E P. The human KLK8 (neuropsin/ovasin) gene: identification of two novel splice variants and its prognostic value in ovarian cancer. Clin Cancer Res 2001;7:806-811.

32. Luo L-Y, Katsaros D, Scorilas A, Fracchioli S, Piccinno R, Rigault de la Longrais I A, Howarth D J C, Diamandis E P. Prognostic value of human kallikrein 10 expression in epithelial ovarian carcinoma. Clin Cancer Res. 2001;7:2372-2379.

33. Yousef G M, Scorials A, Jung K, Ahsworth L K, Diamandis E P. Molecular cloning of the human kallikrein 15 gene (KLK15): Up-regulation in prostate cancer. J Biol Chem 2001;276:53-61.

34. Luo L Y, Rajpert-De Meyts E, Jung K, Diamandis E P. Expression of the normal epithelial cell-specific I (NES1; KLK10) candidate tumor suppressor gene in normal and malignant testicular tissue. Br J Cancer 2001 ;85:220-224.

35. Yousef G M, Magklara A, Chang A, Jung K, Diamandis E P. Cloning of a new member of the human kallikrein gene family, KLK14, which is down-regulated in different malignancies. Cancer Res 25 2001;61:3425-3431.

36. Tanimoto H, Underwood U, Shigemasa K, Yan Y, Clarke J, O'Brien T J. The stratum corneum chymotryptic enzyme that mediates shedding and desquamation of skill cells is highly over-expressed in ovarian tumor cells. Cancer 1999;86:2074-2082.

37. Underwood L J, Tanimoto H, Wang Y, Shigemasa K, Parmley T H, O'Brien T J. Cloning of tumor-30 associated differentially expressed gene-14, a novel serine protease overexpressed by ovarian carcinoma. Cancer Res 1999;59:4435-4439.

38. Campbell A M. Production and purification of antibodies. In: Immunoassay (Diamandis E P, Christopoulos T K, Eds.), pp 95-115. Academic Press, San Diego, Calif., 1996.

39. Magklara A, Grass L, Diamandis E P. Differential steroid hormone regulation of human glandular kallikerin 35 (hK2) and prostate specific antigen (PSA) in breast cancer cell lines. Breast Cancer Res Treat 2000,59:263-270.

40. Ferguson R A, Yu H, Kalyvas M, Zammit S, Diamandis E P. Ultrasensitive detection of prostate specific antigen by a time-resolved immunofluorometric assay and the Immulite Immunochemiluminescent third generation assay: potential applications in prostate and breast cancers. Clin Chem 1996;42:675-684.

41. Black M H, Magklara A, Obiczu C V, Melegos D N, Diamandis E P. Development of an ultrasensitive immunoassay for human glandular kallikrein with no cross-reactivity from prostate-specific antigen. Clin Chem 1999;45:790-799.

42. Stenman U H, Leinonen J, Alfihan H, Rannikko S, Tuhkanen K, Alfthan O. A complex between prostate-specific antigen and alpha 1-antichymotrypsin is the major form of prostate-specific antigen in serum of patients with prostatic cancer: assay of the complex improves clinical sensitivity for cancer. Cancer Res 1991;51 :222-226.

43. Christensson A, Laurell C B, Lilja H. Enzymatic activity of prostate-specific antigen and its reaction with extracellular serine proteinase inhibitors. Eur J Biochem 1990; 194:755-763.

44. Diamandis E P. Prostate specific antigen—its usefulness in clinical medicine. Trends Endocrinol Metab 50 1998;9:310-316.

45. Luo L Y, Grass L, Diamandis E P. The normal epithelial cell-specific I (NES I) gene is un-regulated by steroid hormones in the breast carcinoma cell line BT-474. Anticancer Res 2000;20:981-986.

46. Lilja H. A kallikrein-like serine protease in prostatic fluid cleaves the predominant seminal vesicle protein. J Clin Invest 1985;76:1899-1903.

47. Lovgren J, Rajakoski K, Karp M, Lundwall A, Lilja H. Activation of the zymogen form of prostate-specific antigen by human glandular kallikrein 2. Biochem Biophys Res Commun 1997;238:549-555.

48. Kumar A, Mikolajczyk S D, Goel A S, Millar L S, Saedi M S. Expression of pro form of prostate-specific antigen by mammalian cells and its conversion to mature, active form by human kallikrein 2. Cancer Res 1997;57:3111-3114.

49. Takayama T K, Fujikawa K, Davie E W. Characterization of the precursor of prostate-specific antigen-activation by trypsin and by human glandular kallikrein. J Biol Chem 1997;272:21582-21588.

50. Takayama T K, Carter C A, Deng T. Activation of prostate-specific antigen precursor (pro-PSA)by a novel human prostatic serine protease identified by degeneration. Biochemistry 2001;40:1679-1687.

1. A method for screening a subject for ovarian or prostate cancer comprising (a) obtaining a biological sample from a subject; (b) detecting the amount of hK11in said sample; and (c) comparing said amount of hK11 detected to a predetermined standard, where detection of a level of hK11 that is different than that of a predetermined standard indicates disease.

2. A method as claimed in claim 1 wherein the predetermined standard is hK11 levels in samples from a normal control and wherein an increase in hK11 levels in the subject compared to the normal control indicates disease.

3. A method as claimed in claim 1 which further comprises in step (b) detecting one or more of human stratum corneum chymotryptic enzyme (HSCCE), kallikrein 4, KLK4 gene, kallikrein 6, kallikrein 8, KLK8 gene, kallikrein 9, KLK9 gene, kallikrein 10, KLK10 gene, CA125, CA15-3, CA19-9, OVX1, lysophosphatidic acid (LPA) or carcinoembryonic antigen (CEA).

4. A method as claimed in claim 1 which further comprises in step (b) detecting one or more of kallikrein 2, KLK5 gene, HER-2, KLK10 gene, KLK15 gene, and prostate-specific antigen.

5. A method of monitoring the stage of prostate or ovarian cancer in a subject suffering from prostate or ovarian cancer which comprises (a) identifying a subject suffering from prostate or ovarian cancer; (b) determining hK11 levels in a biological sample from the subject to establish a baseline hK11 level for the subject; (c) measuring hK11 levels in biological samples of the same type from the subject at subsequent time periods; and (d) comparing the measured hK11 levels with the baseline hK11 levels, wherein an increase in measured hK11 levels in the subject versus baseline hK11 levels in the subject is associated with a cancer which is progressing and a decrease in measured hK11 levels versus baseline hK11 levels is associated with a cancer which is regressing or in remission.

6. A method as claimed in claim 1 wherein the hK11 is measured using an antibody specific for hK11.

7. A method for detecting the presence of prostate or ovarian cancer in a subject suspected of suffering from prostate or ovarian cancer which comprises (a) measuring transcription levels of hK11 in a biological sample from a subject suspected of suffering from prostate or ovarian cancer; and (b) comparing the measured transcription levels of hK11 with hK11 transcription levels in a biological sample from a predetermined standard.

8. A method for detecting prostate or ovarian carcinoma in a subject by measuring hK11 in a sample from the subject comprising:
(a) contacting a biological sample from the subject with an antibody specific for hK11 which is directly or indirectly labelled with a detectable substance;
(b) detecting the detectable substance to measure hK11 in the sample;
(c) comparing the measured level of hK11 to levels obtained for a predetermined standard.

9. A method for detecting prostate or ovarian cancer in a subject comprising:
(a) reacting a biological sample from the subject with a first antibody specific for hK11 which is directly or indirectly labeled with a detectable substance, and a second antibody specific for hK11 which is immobilized;
(b) separating the first antibody from the second antibody to provide a first antibody phase and a second antibody phase;
(c) detecting the detectable substance in the first or second antibody phase thereby measuring hK11 in the biological sample; and
(d) comparing the measured hK11 with levels measured for a predetermined standard.

10. A method as claimed in claim 1 wherein the biological sample is serum.

11. A method as claimed in claim 8 wherein the predetermined standard corresponds to hK11 levels in samples from healthy control subjects or from other samples of the subject.

12. A method as claimed in claim 1 wherein detection of an amount of hK11 greater than that in the predetermined standard indicates disease, or an increased risk of disease progression.

13. A method as claimed in claim 9 wherein in step (a) the first and second antibodies are contacted simultaneously or sequentially with the biological sample.

14. A method as claimed in claim 6 wherein the antibody is a monoclonal antibody, a polyclonal antibody, immunologically active antibody fragments, a humanized antibody, an antibody heavy chain,. an antibody light chain, a genetically engineered single chain Fᵥ molecule, or a chimeric antibody.

15. A method as claimed in claim 8 wherein the detectable substance is alkaline phosphatase.

16. A method as claimed in claim 15 wherein the alkaline phosphatase is detected using a fluorogenic substrate.

17. A method as claimed in claim 16 wherein the fluorogenic substrate is 4-methylumbelliferyl phosphate, 5-fluorosalicyl phosphate, or diflunisal phosphate.

18. A method as claimed in claim 16 wherein hK11 is measured using time-resolved fluorescence.

19. A method for detecting prostate or ovarian cancer comprising (a) reacting a biological sample from a subject with an antibody specific for hK11 which is directly or indirectly labelled with an enzyme; (b) adding a substrate for the enzyme wherein the substrate is selected so that the substrate, or a reaction product of the enzyme and substrate forms fluorescent complexes; (c) quantitating hK11 in the biological sample by measuring fluorescence of the fluorescent complexes; and (d) comparing the quantitated levels to levels obtained for a predetermined standard.

20. A method as claimed in claim 19 wherein the enzyme is alkaline phosphatase and the substrate is 4-methylumbelliferyl phosphate, 5-fluorosalicyl phosphate, or diflunisal phosphate.

21. A method for imaging a tumor associated with hK11 comprising:
(a) incubating the tumor with an agent that binds to hK11 for a sufficient period of time to permit the agent to bind to hK11associated with the tumor, where the agent carries a label for imaging the tumor; and
(b) detecting the presence of the label localized to the tumor.

22. A method as claimed in claim 21 which further comprises in step (a) incubating with one or more of an agent that binds to human stratum corneum chymotryptic enzyme (HSCCE), kallikrein 4, kallikrein 5, kallikrein 6, kallikrein 8, kallikrein 9, kallikrein 10, kallikrein 15, CA125, CA15-3, CA19-9, OVX1, lysophosphatidic acid (LPA) or carcinoembryonic antigen (CEA).

23. A method as claimed in claim 22 wherein each agent is labeled so that it can be distinguished in step (b).

24. A method as claimed in claim 21 wherein the agent is an antibody which recognises hK11.

25. A method as claimed in claim 21 wherein the label is a radiolabel, fluorescent label, nuclear magnetic resonance active label, positron emitting isotope detectable by a positron emission tomography (“PET”) scanner, chemiluminescer, or enzymatic marker.

26. A kit for carrying out a method as claimed in claim 1.

27. A kit for carrying out a method as claimed in claim 1 comprising an antibody specific for hK11 labeled with an enzyme; and a substrate for the enzyme.
